(19) United States
(12) Patent Application Publication
(10) Pub. No.: US 2026/0306688 A1
(43) Pub. Date: Oct. 8, 2026

(54) Medical Instrument and Retainer and Tip Protection Sheath

(71) Applicant: Cygnus Medical, LLC, Branford, CT (US)

(72) Inventor: Shaun Sweeney, Wayne, NJ (US)

(21) Appl. No.: 19/467,607

(22) Filed: Feb. 2, 2026

Related U.S. Application Data
(63) Continuation-in-part of application No. 17/963,617, filed on Oct. 11, 2022, now Pat. No. 12,539,189.
(60) Provisional application No. 63/254,359, filed on Oct. 11, 2021.

(51) Int. Cl. A61L 2/26; A61B 17/3201; A61B 90/00; A61L 2/07; A61L 2/206; A61L 103/15

(52) U.S. Cl. CPC A61L 2/26; A61B 17/3201; A61B 90/08; A61L 2/07; A61L 2/206; A61B 2090/0814; A61L 2103/15; A61L 2202/181

(57) ABSTRACT

A surgical instrument, tip protection sheath, and retainer for positioning of medical instrument for cleaning and sterilization. The surgical instrument may be surgical scissors or forceps, having two legs hinged together, each having a ring handle at one end thereof and a tool end at another end thereof. The tip protection sheath is a trapezoid shaped sleeve open along an upper edge of the trapezoid. The surgical instrument tool end is inserted into the open upper edge of the trapezoid. A retainer holds the ring handles of the surgical instrument to hold the surgical instrument tool ends in an open position within the sheath.

### FIELD OF THE INVENTION

The invention relates to the field of medical devices, and in particular, the sterilization of surgical instruments. Specifically, the invention relates to methods of securing surgical instruments in an opened position within a sterilization container, a retainer for securing surgical instruments in an opened position within a sterilization container, and a sterile package containing a sterilized surgical instrument.

### BACKGROUND OF THE INVENTION

Surgical instruments are commonly available as single-use, disposable instruments or multiple-use, reusable instruments. In many cases, a single-use, disposable instrument is more cost-effective and sanitary than a multiple-use, reusable instrument. However, some categories of instruments, particularly surgical scissors and heavy cutting instruments, are more cost-effective, and more effective tools for the surgeon, when constructed as a reusable instrument.

A safety concern with any reusable instrument is sterility, and the need to prevent cross-contamination between patients. Each surgical operation leaves biological residue from the patient's body on the surgical instruments. Medical professionals at hospitals and other surgical facilities have long been aware of the risk that small fragments of tissue from a prior case may remain on a grasping platform or cutting blades and have developed protocols to remove biological residue and then to sterilize the surgical instruments.

Notably, it is very important that every surface of a surgical instrument be cleaned to remove biological residues, and then sterilized after the biological residues are removed. Cleaning surgical instruments after a procedure requires specialized equipment and techniques. Common medical washing systems use hot water and surfactants formulated for removal of biological materials. The most common medical sterilization system is a steam sterilizer, also known as an autoclave, which uses saturated steam at 121-132° C. for a typical time period of 30 minutes under a pressure of 1 atm once all surfaces have reached a temperature of 121° C. Another common system is a vaporized hydrogen peroxide system.

Although traditionally instruments would be sterilized in a steam sterilizer and then packaged as needed until use, the current preferred packaging is a “peel pouch,” also called a “peel pack.” A peel pouch is a disposable sterilization package which has at least one gas permeable wall (such as medical grade paper, or Tyvek®) and one transparent wall (e.g. a clear plastic film) and which receives the cleaned instrument, is then sealed, and then sent to the steam sterilizer or other system. The steam or other sterilizing agent permeates the peel pouch and the instrument is sterilized. The instrument then remains in and is stored in the sterile peel pouch until needed for use, whereupon the sterile peel pouch may be opened by an operating room nurse and the sterile instrument provided to the surgeon for use. The peel pouch has become popular as it provides a greater assurance that the instrument has not become contaminated during handling and storage.

An AST medical sterilization standard requires that hinged instruments and instruments with ratchets should be held in an open position during cleaning and sterilization to allow the cleaning solutions and sterilizing agents to reach all bladed edges, serrations, ratchets and joints on such instruments.

There are a number of known devices, such as racks, stringers and clamps, designed to be used to hold an instrument in an open or unlatched position during cleaning and sterilization. For example, some sterilization systems use stainless steel instrument stringers, such as sold by Key Surgical, which are I-shaped bars or U-shaped rings which have parallel spaced apart rods that are threaded through the ring handles of surgical scissors to retain the scissors in an open position. Other systems use metal instrument racks that have upwardly extending I-shaped bars onto which the handles of the surgical instruments are inserted to keep the instruments in an opened position. Band clamps, such as sold by Healthmark Industries under ProSys™ brand, are made with stainless steel and are inserted through the loops and rings of the instrument handle to keep the instrument open.

The known holding devices, however, suffer from a number of drawbacks. The conventional devices are typically designed to hold multiple surgical instruments at once. This may have a negative effect on sterilization efficacy as instrument surfaces are touching during sterilization and may not be properly exposed to cleaning agents and sterilants.

More significantly, the commonly used racks and stringers are not compatible with single use sterilization peel pouches. It is not possible to have parallel spaced apart rods that are threaded through the ring handles of surgical scissors to retain the scissors in an open position if each set of scissors is sealed in a peel pouch. In the absence of some type of holding device however, there is the risk that the blades of scissors or other instrument parts may shift to a closed position, either due to gravity, or pressure from container sidewalls when the peel pouches are loaded in a steam sterilizer, or pressure of an adjacent peel pouch containing another instrument, or due to pressure from manual or automated handling of the peel pouch during sterilization.

Furthermore, the conventional devices are typically made with stainless steel and can be bulky and rather expensive. They also require cleaning and sterilization after each use, thus adding an extra step to the process. Conventional clamps can be cumbersome to insert into the instruments and add weight to sterilization peel pouches making it harder to keep the pouches in correct position before, during and after sterilization.

Also, in some cases, known holding devices are not useful if the cleaning and/or sterilization method involves use of a liquid bath and the bath size is not compatible with the holding devices.

There exists a need in the medical field for a retainer for securing surgical instruments in an opened position within a sterilization container that is light and easy to handle and operate. There further exists a need for a retainer that is inexpensive and disposable, and can be used with various sterilization systems including peel pouches.

### SUMMARY OF THE INVENTION

To solve the problems of the conventional sterilization devices and to achieve the above-stated objectives, the present invention provides a surgical instrument, tip protection sheath, and retainer for positioning of medical instrument for cleaning and sterilization. The surgical instrument may be surgical scissors or forceps, having two legs hinged together, each having a ring handle at one end thereof and a tool end such as scissors blades or forceps jaws at another end thereof. The tip protection sheath is a trapezoid shaped sleeve open along an upper edge of the trapezoid. The surgical instrument tool end is inserted into the open upper edge of the trapezoid. A retainer holds the ring handles of the surgical instrument to hold the surgical instrument tool ends in an open position within the sheath.

The retainer has an elongated body portion with a first end and a second end, and a first end section positioned at the first end and a second end section positioned at the second end. Each of the first end section and the second end section include a central opening such that the end sections have a generally ring-like structure. Each ring-like structure of the first and second end sections has a gap therein. The gap and the central opening are configured to receive and retain a ring handle of a surgical instrument.

In some embodiments, the retainer is made with material which is opaque to X-rays.

In certain embodiments, the elongated body has a length of about 1 inch to 3.5 inches. In some preferred embodiments, the elongated body has a length of about 1.9 inches. In additional preferred embodiments, the elongated body has a length of about 2.7 inches.

In some cases, the gap has a tapered shape that tapers from an outer surface to an inner surface of the first and second end sections.

In some embodiments, the retainer is made of polyurethane material.

In certain embodiments, the elongated body with the first end section and the second end section are made as a unitary piece.

The surgical instrument may comprise surgical scissors wherein the tool ends thereof will comprise scissors blades. Alternatively, the surgical instrument may comprise surgical forceps and the tool ends thereof will comprise forceps jaws.

To further solve the problems of the conventional sterilization devices and to achieve the above-stated objectives, the present invention provides a method of sterilizing a surgical instrument having two legs hinged together, each having a ring handle at one end thereof and a tool end at another end thereof, comprising the steps of: placing the surgical instrument tool ends in a trapezoid tip protection sheath having a gas permeable wall and a transparent wall, the gas permeable wall and a transparent wall being affixed to each other along a base edge and two side edges of the trapezoid, but not along an upper edge of the trapezoid, whereby the sheath has an opening to the sheath at the upper edge of the trapezoid and a space within the sheath, wherein the surgical instrument tool ends are inserted into the opening and received within the space within the sheath; positioning the surgical instrument tool ends in an open position with the blades separated from each other; securing the surgical instrument tool ends in the open position using a retainer having an elongated body with a first end and a second end, the first end and second end having a central opening and a gap, by inserting one ring handle of the surgical instrument through the gap and into the central opening of the first end and inserting another ring handle of the surgical instrument through the gap and into the central opening of the second end; and placing the surgical instrument, tip protection sheath, and retainer into a sterilization unit and performing a sterilization process.

The method further includes placing the surgical instrument with the retainer in a sterilization container and performing a sterilization process.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

FIG. 1 is a perspective view of the retainer of the surgical instrument, tip protection sheath, and retainer for positioning of medical instrument for cleaning and sterilization of the present invention.

FIG. 2 is a top view of the retainer of FIG. 1.

FIG. 3 is a front view of the retainer of FIG. 1.

FIG. 4 is a back view of the retainer of FIG. 1.

FIG. 5 is a perspective view of another embodiment of the retainer of the surgical instrument, tip protection sheath, and retainer for positioning of medical instrument for cleaning and sterilization of the present invention.

FIG. 6 illustrates the retainer of FIG. 1, positioned on a surgical instrument, in particular, a first embodiment of surgical scissors.

FIG. 7 illustrates the retainer of FIG. 1, positioned on a surgical instrument, in particular, a second embodiment of surgical scissors, placed in a sterilization pouch.

FIG. 8 illustrates the retainer of FIG. 1, positioned on a surgical instrument, in particular, surgical forceps, placed in a sterilization pouch.

FIG. 9 illustrates a tip protection sheath for use in the surgical instrument, tip protection sheath, and retainer for positioning of medical instrument for cleaning and sterilization of the present invention.

FIG. 10 illustrates two of the tip protection sheaths of FIG. 9 attached to each other with a tearaway connection.

FIG. 11 illustrates a surgical instrument, in particular, a third embodiment of surgical scissors.

FIG. 12 illustrates the surgical scissors of FIG. 11 with the tip protection sheath of FIG. 9 positioned on the scissor blades.

FIG. 13 illustrates the retainer of FIG. 1, positioned on a surgical instrument, in particular, a fourth embodiment of surgical scissors, with the tip protection sheath of FIG. 9 positioned on the scissor blades.

FIG. 14 illustrates the retainer of FIG. 1, positioned on a surgical instrument, in particular, the first embodiment of surgical scissors of FIG. 6, with the tip protection sheath of FIG. 9 positioned on the scissor blades.

### DETAILED DESCRIPTION OF THE INVENTION

The embodiments of the present invention will be described with reference to the accompanying drawings and below description.

The term “about” as used in connection with a numerical value throughout the specification and the claims denotes an interval of accuracy, familiar and acceptable to a person skilled in the art. In general, such interval of accuracy is ±10%. Thus, “about ten” means 9 to 11. All numbers in this description indicating sizes, amounts, ratios of materials, physical properties of materials, and/or use are to be understood as modified by the word “about,” except as otherwise explicitly indicated.

The AST Standards of Practice for the Decontamination of Surgical Instruments state that hinged instruments and instruments with ratchets should be in the open position during cleaning and sterilization process. The present invention provides an easy and efficient way for securing surgical instruments in an opened position for sterilization.

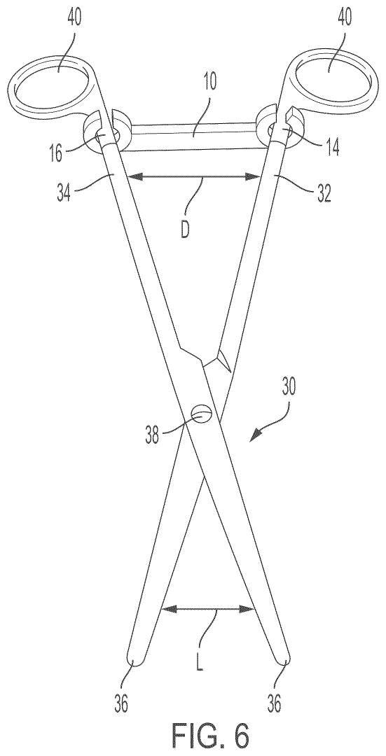

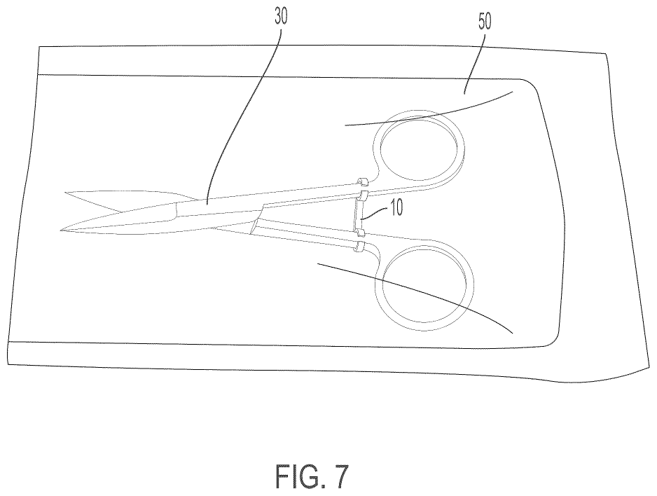

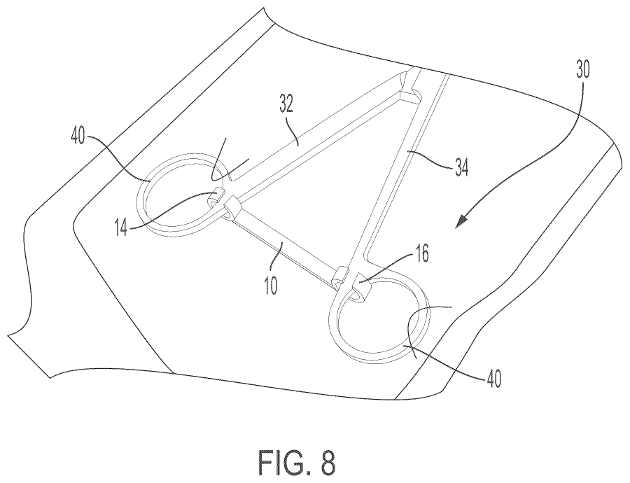

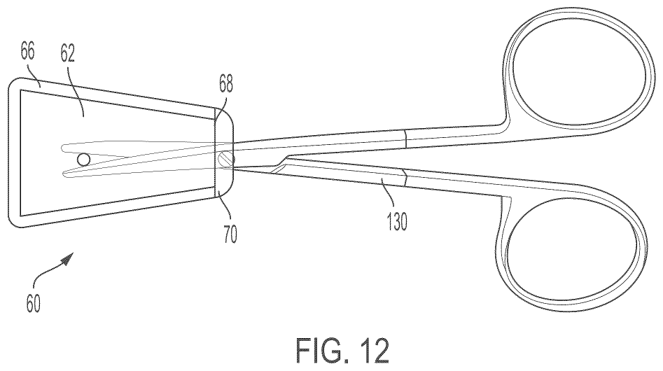

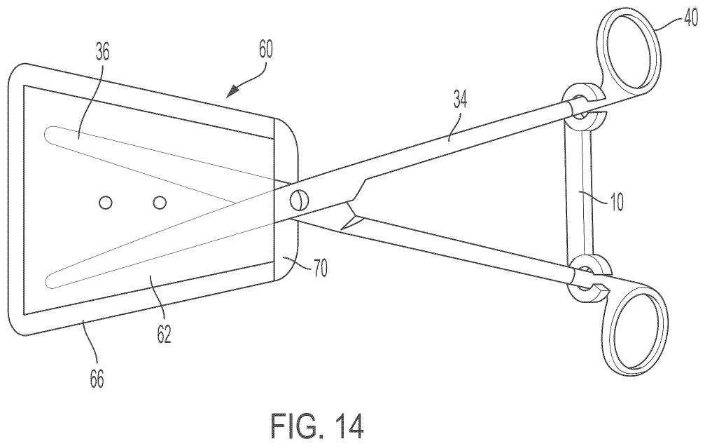

Referring now to FIGS. 6-11 and 11-14, A surgical instrument, tip protection sheath, and retainer, comprises a surgical instrument 30, 130, or 230 having two legs hinged together. The two legs each have a ring handle 40, 140, 240 at one end thereof and a tool end at the other end thereof. The surgical instrument may surgical scissors and the tool ends thereof may comprise scissors blades as seen in FIGS. 6-7 and 11-14. Or they may be forceps as illustrated in part in FIG. 8, and the tool ends thereof may comprise forceps jaws.

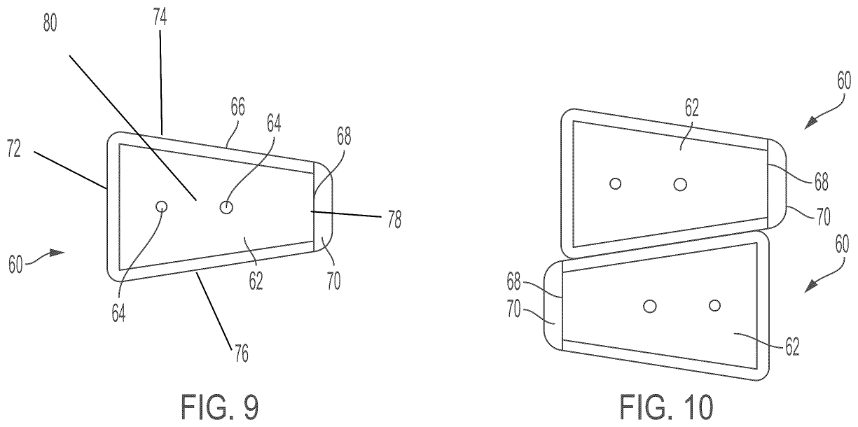

Referring now to FIGS. 9-10 and 12-14, a tip protection sheath 60 is a trapezoid shaped envelope. Sheath 60 has a gas permeable wall 66 and a transparent wall 62. Gas permeable wall optionally includes perforations 64 in the center of the wall. The gas permeable wall 66 and transparent wall 62 are affixed to each other along the perimeter of the trapezoid except for the upper edge 78. The gas permeable wall 66 and transparent wall 62 are welded together along the base edge 72 and two side edges 74, 76 of the trapezoid. The gas permeable wall 66 and transparent wall 62 are not welded together along the upper edge 78 of the trapezoid, to provide an opening 68 to the sheath 60 at the upper edge 78 of the trapezoid and a space 80 within the sheath 60. Preferably, the gas permeable wall 66 has an upper edge which extends beyond the transparent wall to form an apron 70. The surgical instrument tool end such as the surgical scissors blades 36, 136, 236 of the surgical scissors 30, 130, 230 are inserted into the opening 68 and received within the space 80 within the sheath 60.

A retainer 10 has an elongated body 12 with a first end 14 and a second end 16. The first end and second end 14 and 16 have a central opening 18 and a gap 20, whereby the ring handles 40, 140, 240 of the surgical instrument are received within the gap 20 and said central opening 18 to position and retain the surgical instrument tool ends 36, 136, 236 within the space within the sheath in an open position.

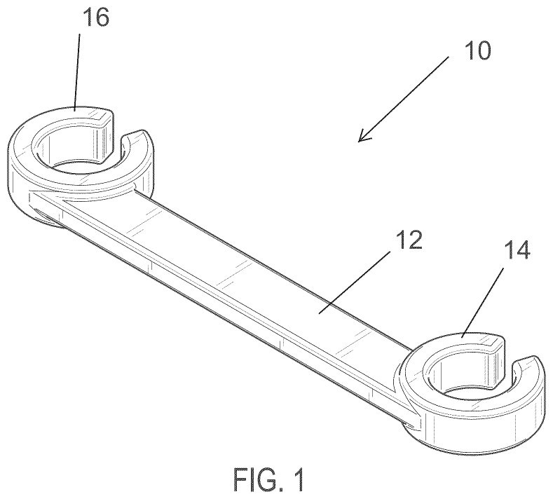

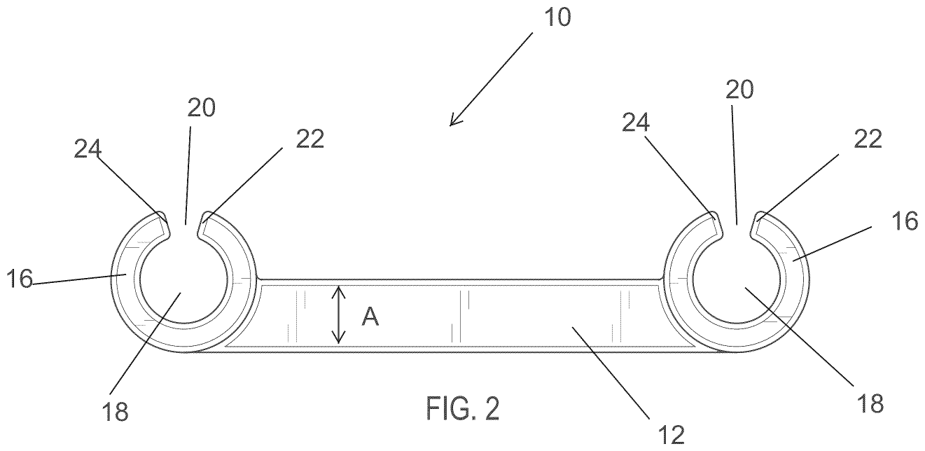

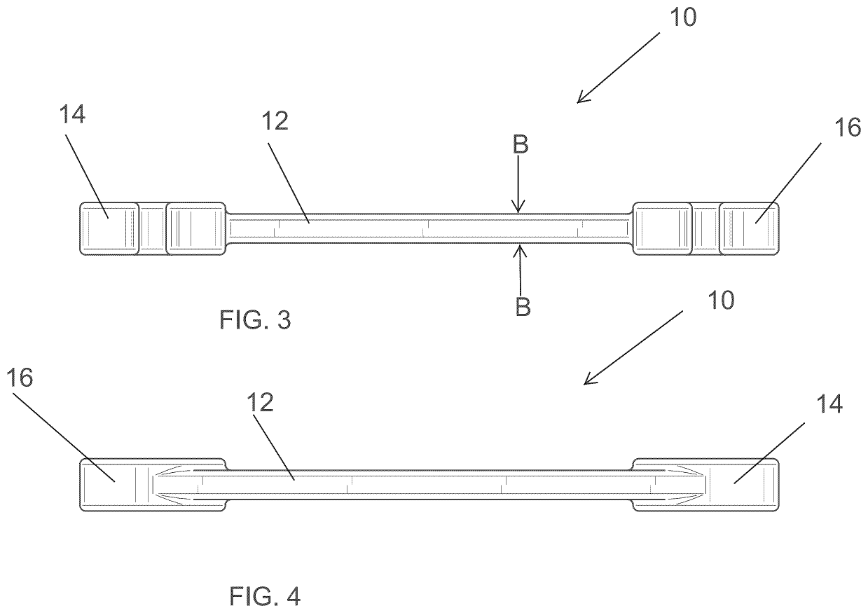

As illustrated in FIG. 1, the retainer 10 includes an elongated body section 12 and two end sections 14, 16 positioned on each end of the body section 12. The body section 12 has a height “A” shown in FIG. 2 and a width “B” shown in FIG. 3. In the exemplary embodiment shown in FIGS. 1-4, the body section 12 has a flattened bar shape with the height A that is larger than the width B. It is understood that the elongated body section can have other suitable shapes, such as, e.g., a rod shape, a square or triangle cross-sectioned bar shape, or a bar shape with a width larger than a height.

The retainer 10 of the invention is preferably of single-piece construction, which is composed of a single material or blend of materials. Generally, the material used to fabricate the retainer 10 must have a sufficient structural strength to hold a surgical instrument open, have a resilience so that the retainer can snap on and off a surgical instrument, be stable to steam sterilization and other common sterilization methods, and desirably is disposable and/or recyclable. Preferably, the materials used for the retainer 10 can withstand temperatures of about 250 to 300 degrees Fahrenheit during sterilization. In general, the preferred material for retainer 10 is a polymeric material. Examples of suitable materials include but are not limited to polyurethane, polypropylene, fluoropolymers, polyetherimide, polyether ether ketone (PEEK), and other suitable plastics. In one preferred embodiment, the retainer is made with polyurethane material. The retainer 10 of the invention can be manufactured using conventional tool and die equipment, which is readily available in the manufacturing field for molding, cutting and shaping materials. Preferably, the material used for the retainer 10 is latex free to accommodate users and/or patients that may have an allergy to latex.

The materials used for the retainer of the invention preferably contain additives that make the material X-ray detectable. Surgery often requires the placement of foreign material inside the human body. Many systems and safeguards are applied in the operating room to ensure that no material is left behind unintentionally after closure. One of such methods is performing an X-ray of the body at and near the surgical site to ensure that no surgical instruments are left inside the body. The X-ray will detect any radiopaque/X-ray detectable foreign materials, which can be removed prior to closing the surgical opening. Thus, using an X-ray detectable material in the retainer of the invention ensures that the retainer is not accidentally left in a patient's body during surgery. Any of the known suitable radio-opaque biocompatible materials may be used in accordance with the invention, including, for example, barium sulfate, bismuth oxide, bismuth halide, or a halogen-containing plasticizer, diol, or other such halogen-containing material. In some advantageous embodiments, barium sulfate, bismuth oxide, is used.

As shown in FIGS. 1-4, the retainer 10 has two end sections 14 and 16 positioned on the opposite ends of the body 12. In the embodiment shown in these figures, each end section has a circular shape with a central opening 18. The central opening 18 also has a circular shape, such that, in the preferred embodiment, end sections 14 and 16 form a generally ring-like structure. The central opening 18 is sized so that it can receive a portion of a surgical instrument, such as the ring handles of scissors or forceps. Each end section 14 and 16 has a gap 20 in the ring-like structure whereby the end section can be attached to the portion of a surgical instrument by passing the surgical instrument through the gap 20.

As shown in FIG. 2, each of the gaps 20 has a tapered shape that tapers from a larger separation at the outer circumference of the end sections 14 and 16 to a smaller separation at the inner circumference of the end sections 14 and 16, In other words, the gap 20 is wider on top and narrower at bottom. This allows for an easier insertion of portions of surgical instruments into the gap 20 and then into central opening 18 of the end sections 14, 16, with less material resistance.

An outer radius of the end sections 14 and 16 and a radius of the central opening 18 therein are selected such that the end sections 14 and 16 are rigid enough to remain attached to a portion of a surgical instrument but flexible enough to accommodate portions of surgical instruments with varying diameter and outer shapes. In some advantageous embodiments, an outer radius of the end sections 14 and 16 is about 0.1 to about 0.4 inches. In further advantageous embodiments, a radius of the central opening 18 of each end section 14, 16 is about 0.05 to 0.3 inches. In one preferred embodiment, an outer radius of the end sections 14 and 16 is about 0.25 inches and a radius of the central opening 18 of each end section 14, 16 is about 0.15 inches.

An average width of the gap 20 is selected such that gap 20 permits a portion of a surgical instrument to be inserted through the gap 20 into the central opening 18. Preferably, the average width of the gap 20 is the same or slightly smaller than a width or outer diameter of the portion of the surgical instrument inserted therein. This way, the instrument can be securely retained within the retainer during cleaning/sterilization without falling out of it due to shifting before or during the process. In some advantageous embodiments, the width of the gap 20 is about 0.05 to 0.3 inches. In one preferred embodiment, the width of the gap 20 is around 0.1 inches.

The overall dimensions of the retainer can vary in accordance with dimensions of specific surgical instruments to be inserted in the device. For example, a length of the elongated body 12 of the retainer is chosen based on the size and type of surgical instruments.

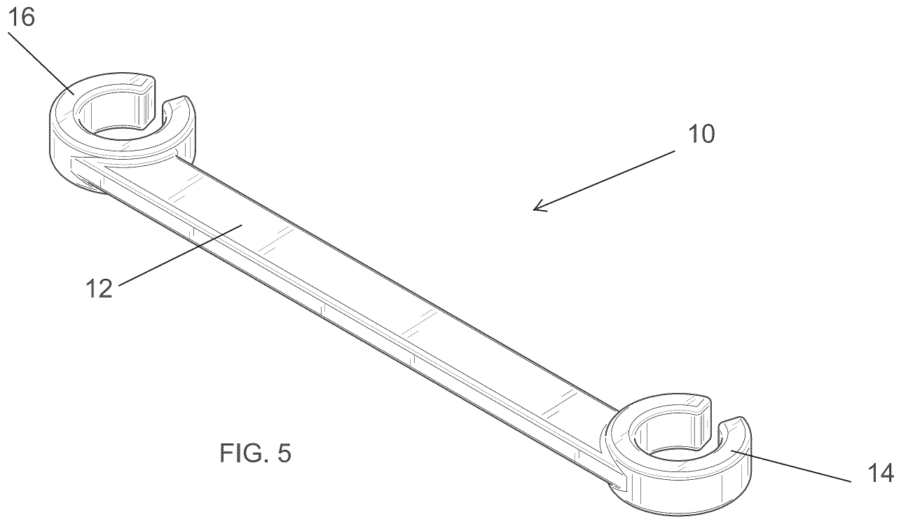

There are two general types of surgical instruments that need to be held in open position during cleaning and sterilization-ratcheted instruments and non-ratcheted hinged instruments. Ratcheted instruments, such as clamps, retractors, etc., have a releasable ratchet mechanism that self-locks the instruments in a closed position. Hinged instruments, such as scissors, etc., have a middle hinge that allows articulation of the instrument legs between open and closed positions, typically via ring-handles. Each of these types of instruments also include instruments of different sizes that depend on a type of procedure the instruments are used for. In some preferred embodiments, the length of the elongated body 12 of the retainer is about 0.5 to 5 inches. In some more preferred embodiments, the length of the elongated body is about 1.5 to 3 inches. In some particularly preferred embodiments, the length of the elongated body is about 1.9 inches, as shown in FIG. 1. In some additional particularly preferred embodiments, the length of the elongated body is about 2.7 inches, as shown in FIG. 5. The height A and the width B of the elongated body 12 of the retainer can also vary depending on the length and other device characteristics.

FIG. 6 illustrates the retainer 10 mounted to a surgical instrument 30, i.e., scissors. The instrument 30 has two movable legs 32 and 34, connected together via a hinge 38. Portions of the legs 32, 34 distal to the hinge terminate with two ring handles 40 and portions of the legs 32, 34 proximal to the hinge form the cutting surfaces or jaws 36. As shown in this figure, the portions of the instrument legs 32, 34 distal to the hinge but proximal to the ring handles 40 are inserted into the central opening 18 of the end sections 14 and 16 of the retainer to keep the instrument in an opened position such that the jaws 36 are separated and can be properly cleaned and sterilized. The length “D” of the elongated body of the retainer 10 between the end sections 14, 16 is chosen to achieve the necessary separation distance “C” between the jaws 36 of the instrument.

Preferably, the end sections 14 and 16 of the retainer 10 are attached to the ring handles 40 of the surgical instrument 30, as illustrated in FIG. 8. In such case, the end sections 14 and 16 receive portions of the ring handles 40 that are adjacent and facing each other. This positioning of the retainer achieves the best results in securely retaining the retainer on the surgical instrument as it prevents the retainer from shifting or sliding along the movable legs before and/or during cleaning/sterilization.

It is understood that the retainer 10 may be positioned at various desired locations on the surgical instrument, e.g., along the movable legs or in the ring handles. The length “D” of the retainer 10 may be selected depending on where the retainer will be positioned on the surgical instrument.

FIG. 6 illustrates the retainer 10 with a surgical instrument 30, i.e., surgical scissors, inserted therein. The instrument 30 has two movable legs 32 and 34, connected together via a hinge 38. Portions of the legs 32, 34 distal to the hinge terminate with two ring handles 40 and portions of the legs 32, 34 proximal to the hinge form the cutting surfaces or jaws 36. As shown in this figure, the portions of the instrument legs 32, 34 distal to the hinge but proximal to the ring handles 40 are inserted into the central opening 18 of the end sections 14 and 16 of the retainer to keep the instrument in an opened position such that the jaws 36 are separated and can be properly cleaned and sterilized. The length “D” of the elongated body of the retainer 10 between the end sections 14, 16 is chosen to achieve the necessary separation distance “C” between the jaws 36 of the instrument.

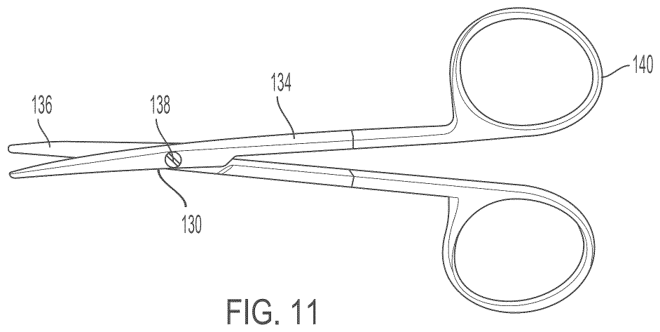

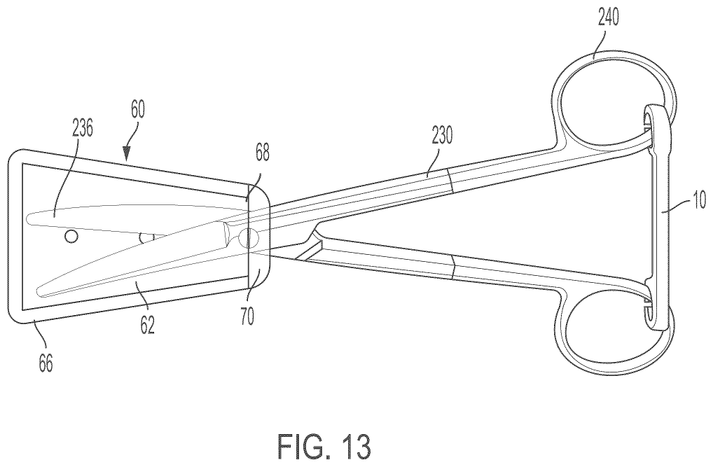

FIGS. 11-12 and 13 illustrate other embodiments of surgical instruments, specifically, surgical scissors 130 in FIGS. 11-12 and 230 in FIG. 13. Like the surgical instrument 30, the surgical scissors 130, 230 include cutting surfaces or jaws 136, 236, a hinge 138, and ring handles 140, 240.

Cleaning of a soiled surgical instrument may begin with a user attaching the retainer 10 to the surgical instrument 30, as shown in FIGS. 6, 7 and 8, to hold the legs 32, 34 in an open position with the legs 32, 34 separated. Next, the user may place the instrument with the retainer into a cleaning/washing unit and operate the unit to clean off bodily fluids and residue from the instrument.

After cleaning, sterilization may take place. If a retainer 10 is attached to the surgical instrument 10, it should be removed prior to the following steps. A method of sterilizing a surgical instrument 30, 130, 230 comprises placing the surgical instrument tool ends 36, 136, 236 in the trapezoid tip protection sheath 60. As noted above, the sheath 60 has an opening 68 at the upper edge of the trapezoid and a space within the sheath. The surgical instrument tool ends 36, 136, 236 are inserted into the opening and received within the space within the sheath. The surgical instrument tool ends 36, 136, 236 are positioned in an open position with the blades separated from each other by manipulating the ring handles 40, 140, 240. The surgical instrument tool ends 36, 136, 236 are then secured in the open position using retainer 10. One ring handle 40, 140, 240 of the surgical instrument is inserted through the gap 20 in retainer 10 and into the central opening 18 of one end of the retainer, then another ring handle 40, 140, 240 of the surgical instrument is inserted through the gap 20 and into the central opening 18 of the second end of the retainer. The surgical instrument 30, 130, 230, tip protection sheath 60, and retainer 10 are placed into a sterilization unit and a sterilization process such as steam or ethylene oxide gas sterilization is performed.

In a preferred embodiment, the surgical instrument 30, 130, 230 with the tip protection sheath 60 and retainer 10 is placed in a peel pouch 50. The pouch 50 is then sealed and then placed in a sterilization unit and exposed to a desired sterilization method, such as steam or ethylene oxide gas sterilization.

It is understood that the retainer of the present invention can be used for keeping surgical instruments open during any type of sterilization process, including for example, dry heat, stead, radiation, and plasma sterilization.

The invention has been described herein above with reference to various and specific techniques and embodiments. It will be understood by one of ordinary skill, however, that reasonable modifications and variations of such embodiments and techniques are possible and can be made without significantly departing from either the spirit or scope of the invention as defined by the claims below.

What is claimed is:

1. A surgical instrument, tip protection sheath, and retainer, comprising:
a surgical instrument having two legs hinged together, each having a ring handle at one end thereof and a tool end at another end thereof;
the tip protection sheath being a trapezoid having a gas permeable wall and a transparent wall, the gas permeable wall and a transparent wall being affixed to each other along a base edge and two side edges of the trapezoid, but not along an upper edge of the trapezoid, to provide an opening to the sheath at the upper edge of the trapezoid and a space within the sheath, the surgical instrument tool end being inserted into the opening and received within the space within the sheath;
the retainer having an elongated body with a first end and a second end, the first end and second end having a central opening and a gap, whereby the ring handles of the surgical instrument are received within the gap and said central opening to position and retain the surgical instrument tool ends within the space within the sheath in an open position.

2. The surgical instrument, tip protection sheath, and retainer of claim 1, wherein the retainer is made with material having radio-opaque qualities.

3. The surgical instrument, tip protection sheath, and retainer of claim 1, wherein the elongated body has a length of about 1 inch to 3.5 inches.

4. The surgical instrument, tip protection sheath, and retainer of claim 1, wherein the gas permeable wall has an upper edge and the transparent wall has an upper edge, and the gas permeable wall has an upper edge which extends beyond the transparent wall upper edge to form an apron.

5. The surgical instrument, tip protection sheath, and retainer of claim 1, wherein the surgical instrument comprises surgical scissors and the tool ends thereof comprise scissors blades.

6. The surgical instrument, tip protection sheath, and retainer of claim 1, wherein the surgical instrument comprises surgical forceps and the tool ends thereof comprise forceps jaws.

7. The surgical instrument, tip protection sheath, and retainer of claim 1, further comprising:
a disposable sterilization package which has at least one gas permeable wall and one transparent wall, wherein the surgical instrument, tip protection sheath, and retainer are contained in the disposable sterilization package.

8. A surgical instrument, tip protection sheath, and retainer, comprising:
surgical scissors having two legs hinged together, each having a ring handle at one end thereof and a blade at another end thereof;
the tip protection sheath being a trapezoid having a gas permeable wall and a transparent wall, the gas permeable wall and a transparent wall being affixed to each other along a base edge and two side edges of the trapezoid, but not along an upper edge of the trapezoid, to provide an opening to the sheath at the upper edge of the trapezoid and a space within the sheath, the surgical scissors blades being inserted into the opening and received within the space within the sheath;
the retainer having an elongated body, with a first end and a second end, the first end and second end having a central opening and a gap, whereby the ring handles of the surgical scissors are received within the gap and said central opening to position and retain the surgical scissors blades within the space within the sheath in an open position.

9. The surgical instrument, tip protection sheath, and retainer of claim 8, wherein the retainer is made with material having radio-opaque qualities.

10. The surgical instrument, tip protection sheath, and retainer of claim 8, wherein the elongated body has a length of about 1 inch to 3.5 inches.

11. The surgical instrument, tip protection sheath, and retainer of claim 8, wherein the gas permeable wall has an upper edge and the transparent wall has an upper edge, and the gas permeable wall has an upper edge which extends beyond the transparent wall upper edge to form an apron.

12. The surgical instrument, tip protection sheath, and retainer of claim 8, further comprising:
a disposable sterilization package which has at least one gas permeable wall and one transparent wall, wherein the surgical instrument, tip protection sheath, and retainer are contained in the disposable sterilization package.

13. A method of sterilizing a surgical instrument having two legs hinged together, each having a ring handle at one end thereof and a tool end at another end thereof, comprising the steps of:
placing the surgical instrument tool ends in a trapezoid tip protection sheath having a gas permeable wall and a transparent wall, the gas permeable wall and a transparent wall being affixed to each other along a base edge and two side edges of the trapezoid, but not along an upper edge of the trapezoid, whereby the sheath has an opening to the sheath at the upper edge of the trapezoid and a space within the sheath, wherein the surgical instrument tool ends are inserted into the opening and received within the space within the sheath;
positioning the surgical instrument tool ends in an open position with the blades separated from each other;
securing the surgical instrument tool ends in the open position using a retainer having an elongated body with a first end and a second end, the first end and second end having a central opening and a gap, by inserting one ring handle of the surgical instrument through the gap and into the central opening of the first end and inserting another ring handle of the surgical instrument through the gap and into the central opening of the second end; and
placing the surgical instrument, tip protection sheath, and retainer into a sterilization unit and performing a sterilization process.

14. The method of claim 13, further comprising placing the surgical instrument, tip protection sheath, and retainer in a sterilization peel pouch prior to placing the surgical instrument, tip protection sheath, and retainer into a sterilization unit and performing the sterilization process.

15. The method of claim 13, wherein the surgical instrument comprises surgical scissors and the tool ends thereof comprise scissors blades.

16. The method of claim 13, wherein the surgical instrument comprises surgical forceps and the tool ends thereof comprise forceps jaws.
